(19) United States
(12) Patent Application Publication
(10) Pub. No.: US 2023/0310307 A1
(43) Pub. Date: Oct. 5, 2023

(54) COMPOSITION FOR USE IN FOOD SUPPLEMENTS AND/OR COSMETIC PRODUCTS, COMPRISING BACILLUS SUBTILIS

(71) Applicant: INSTITUT DR. RILLING HEALTHCARE GMBH, Pliezhausen (DE)

(72) Inventor: Jochen SEIPEL, Pliezhausen (DE)

(21) Appl. No.: 18/024,917

(22) PCT Filed: Sep. 2, 2021
(86) PCT No.: PCT/EP2021/074226

§ 371 Date: Mar. 6, 2023

(30) Foreign Application Priority Data
Sep. 7, 2020 (EP) 20194557.3

(51) Int. Cl. A61K 8/99; A61Q 17/00; A61K 8/25; A61K 8/9789; A61K 8/37; A61K 31/23; A23L 33/115; A23L 33/16; A23L 33/135; A23D 9/02; A23D 9/007

(52) U.S. Cl. CPC A61K 8/99; A61Q 17/005; A61K 8/25; A61K 8/9789; A61K 8/37; A61K 31/23; A23L 33/115; A23L 33/16; A23L 33/135; A23D 9/02; A23D 9/007

(57) ABSTRACT

The present application relates to a composition for a food supplement and/or a cosmetic product comprising:
at least 70% by weight of a vegetable oil and/or fat substance,
at least 0.5% by weight silica, and
at least 0.05% by weight of Bacillus subtilis selected from one of the strains comprising LA13030, CU1, HU58, DE 111, DSM 15544, PXN 21, Rosell-179 and/or DSM 33619.

The present invention relates to a composition comprising Bacillus subtilis, in particular for use in food supplements and/or cosmetic products, a method for producing the composition according to the invention and a food supplement as well as a cosmetic product comprising the composition according to the invention.

Food supplements are becoming increasingly popular, for example to prevent deficiency symptoms. In particular, those based on vegetable oils and/or fats in combination with silicon have a supportive effect in the area of bone metabolism, in the natural synthesis of collagen and elastin, and in strengthening the structure of hair and/or nails.

Silicon is the second most common element on earth and a component of numerous minerals. Silicon never occurs in free form, but mainly as silicon dioxide—also known as silicic acid. The human body contains about 20 mg of silicon dioxide per kilogram of body weight. Since the amount of silicon dioxide decreases with age and the body cannot produce it itself, it must necessarily be ingested with food and/or appropriate food supplements.

The aim of the present invention is therefore to provide a better composition for food supplements and/or for cosmetic products, as well as an improved food supplement and/or an improved cosmetic product.

### DESCRIPTION OF THE INVENTION

According to the invention, the object is achieved by a composition with the features of patent claim 1, a method with the features of patent claim 7, a food supplement with the features of patent claim 9 and a cosmetic product with the features of patent claim 11.

With regard to the composition, this object is achieved by the subject matter of patent claim 1. This composition comprises at least 70% by weight of a vegetable oil and/or fat substance, at least 0.5% by weight of silicic acid, and at least 0.05% by weight of Bacillus subtilis selected from one of the strains comprising LA13030, CU1, HU58, DE 111, DSM 15544, PXN 21, Rosell-179 and/or DSM 33619.

Surprisingly, it has been shown that the proliferation rate of the bacterium Bacillus subtilis selected from one of the strains mentioned could be significantly increased within 24 hours in the presence of silicic acid, which has a particularly advantageous effect on the human immune system.

In the same way, the invention provides a method for preparing the composition according to the invention, comprising the steps:
Supplying at least 70% by weight of a vegetable oil and/or fat substance, at least 0.5% by weight of silicic acid, and at least 0.05% by weight of Bacillus subtilis selected from one of the strains comprising LA13030, CU1, HU58, DE 111, DSM 15544, PXN 21, Rosell-179 and/or DSM 33619, and
mixing the components so that the composition is obtained.

In a further aspect, the present invention also relates to a food supplement and a cosmetic product, comprising the composition according to the invention.

Additional advantageous embodiments of the invention are described in the dependent claims. The features listed individually in the dependent claims can be combined with one another in a technologically meaningful manner and can define further refinements of the invention. In addition, the features specified in the claims are made more precise and explained in more detail in the description, with further preferred configurations of the invention being presented.

The Bacillus subtilis is selected from any of the strains comprising LA13030, which is a German Bacillus subtilis strain; CU1, which is a French Bacillus Subtilis strain; HU58 which is an Indian/English Bacillus subtilis strain; DE 111, which is a US Bacillus subtilis strain; DSM 15544, which is a Japanese Bacillus subtilis strain; PXN 21 which is an English Bacillus subtilis strain; Rosell-179 which is a Canadian bacillus Subtilis strain; and/or DSM 33619. In a particularly preferred embodiment, the composition comprises the Bacillus subtilis strain DSM 33619, which is also known under the designation DSM 21097. When using this strain, it has surprisingly been shown that such a composition, i.e. one comprising at least 70% by weight of a vegetable oil and/or fat substance, at least 0.5% by weight of silicic acid and at least 0.05% by weight of Bacillus subtilis DSM 33619, compared to other strains of Bacillus subtilis, in particular the ATCC 6633 strain, has an antimicrobial effect on various germs.

In a preferred embodiment, the composition can contain Bacillus subtilis in an amount of at least 0.06% by weight, more preferably at least 0.07% by weight, even more preferably at least 0.08% by weight, further preferably at least 0.09% by weight. %, and most preferably at least 0.1% by weight. However, the composition is preferably limited with regard to the maximum content of Bacillus subtilis. Therefore, the composition preferably contains at most 5.0% by weight, preferably at most 2.0% by weight, more preferably at most 1.0% by weight, and most preferably at most 0.5% by weight Bacillus subtilis. For the purposes of the present invention, the term “silicic acid” is basically understood to mean the oligomeric and polymeric condensation products of monosilicic acid Si(OH)₄. Silicic acid can be present both as non-uniformly structured amorphous silicic acid and as crystallized silicic acid. For the purposes of the present invention, the term “silicic acid” also includes so-called precipitated silicic acids. Precipitated silica is understood as meaning aqueous solutions of amorphous silica, so-called silica sols, and acidified, semi-solid silica sols, so-called silica gels. In addition, within the meaning of the present invention, the term “silicic acid” also includes the dried silica gels. Furthermore, for the purposes of the invention, pyrogenic silicic acids are also included in the term “silicic acid.” A fumed silica essentially consists of an amorphous SiO₂powder, which is obtained by flame hydrolysis of silanes or SiCl₄.

In a preferred embodiment, the silicic acid is therefore a precipitated and/or a pyrogenic silicic acid.

In a particularly advantageous embodiment, the composition comprises silicic acid, in particular precipitated, pyrogenic and/or a mixture of these types, in an amount of at least 0.8% by weight, more preferably in an amount of at least 1.0% by weight, even more preferably in an amount of at least 2.5% by weight, further preferably in an amount of at least 2.8% by weight, and most preferably in an amount of at least 7.0% by weight. However, the proportion must not be too high. The silicic acid content is therefore preferably at most 20% by weight, more preferably at most 15% by weight, further preferably at most 10% by weight.

The silicic acid advantageously has a limited water content according to DIN EN ISO 787-2 (1995-04) of at most 3.0% water by weight. However, it is preferably provided that the silicic acid has a water content of 0.1% by weight to 0.5% by weight, more preferably a water content of 0.1% by weight to 0.3% by weight, based in each case on the total amount of silicic acid in the has composition. The silicic acid also preferably has a BET surface area according to DIN ISO 9277 (2014-01) of 150 m²/g to 250 m²/g, in particular from 160 m²/g to 200 m²/g.

In a preferred embodiment of the method according to the invention, the silicic acid is therefore dried before mixing. For this purpose, the silicic acid is exposed to a maximum temperature of 200° C. for at least 10 minutes.

In an advantageous embodiment, the composition comprises at least 80% by weight, preferably at least 90% by weight, more preferably at least 92% by weight, even more preferably at least 97% by weight, and most preferably at least 99% by weight. % of vegetable oil and/or fat substance. In this context, it is particularly preferred that the vegetable oil and/or fat substance is selected from the series comprising olive oil, coconut oil, almond oil, pomegranate seed oil, mango butter, shea butter and/or mixtures thereof.

The vegetable oil and/or fat substance is very particularly preferably selected from the series of medium-chain triglycerides (MCT). In the context of the present invention, medium-chain triglycerides are mixtures which usually contain 50-65% by weight of caprylic acid (C8) and 30-45% by weight of capric acid (C10) as main components. Other ingredients such as caproic acid (C6), lauric acid (C12) and myristic acid (C14) may be present in small amounts. Typical medium-chain triglycerides known from the prior art and commercially available are known, for example, under the trade names Mygliol 812® from Sasol, Myritol 312® from Cognis and Tegosoft® CT from Evonik.

The use of these oils and/or fats in cosmetic products and in food supplements is known in principle to the person skilled in the art. The medium-chain triglycerides are helpful in weight loss, improve mental and physical performance and also protect against various diseases. Because of their structure, medium-chain triglycerides have beneficial physiological properties over traditional long-chain fatty acids, and are therefore easier for the body to absorb. This means that these MOTs do not have to be emulsified in the gut by bile acids in order for them to be better metabolized. They also do not require cleavage by pancreatic lipase, an enzyme in the pancreas. The different compounds of the medium-chain triglycerides are transported directly in the blood, without detouring via the lymphatic system, to the liver, where they are preferentially oxidized compared to conventional fats and can form more ketone bodies, which are used in particular by the heart and brain as energy sources. For these reasons, medium-chain triglycerides are preferably used in clinical nutrition for the dietary treatment of various diseases, such as malabsorption syndrome, lymphangiectasia, Whipple's disease, chylothorax, exocrine pancreatic insufficiency or as part of a ketogenic diet. The ketogenic diet is a carbohydrate-limited, protein- and energy-balanced and therefore high-fat form of dietary nutrition, in which the body obtains its energy requirements only from fat and the glucose substitute produced from it in the body, the eponymous ketone bodies. A ketogenic diet is used as a therapeutic method, especially in children with drug-resistant epilepsy, glucose transporter disorders and pyruvate dehydrogenase deficiency. Recent research results also make therapy for Alzheimer's disease appear possible.

The food supplement or the cosmetic product can comprise the composition according to the invention in an amount of 10 to 100% by weight, more preferably 20 to 100% by weight.

If the food supplement comprises 100% by weight of the composition according to the invention, it is advantageously provided that the composition contains 97.1% by weight MCT oil, 2.8% by weight silica and 0.1% by weight Bacillus subtilis of the MM 40 strain. Furthermore, the food supplement and/or the cosmetic product can include other agents within the meaning of the Food Supplements Ordinance, such as vitamins and minerals, including trace elements.

The vitamins and minerals are advantageously selected from the group comprising vitamin C, vitamin D3, vitamin K, calcium and magnesium salts and mixtures thereof. A particularly preferred vitamin is vitamin C, which is selected from the group comprising L-ascorbic acid, sodium L-ascorbate, calcium L-ascorbate, potassium L-ascorbate, Lascorbyl-6-palmitate and mixtures thereof. Another preferred vitamin is vitamin D in the form of cholecalciferol or ergocalciferol. Other preferred vitamins are vitamin K, preferably as phylloquinone, vitamin B3 and vitamin E.

According to an advantageous embodiment, the food supplement can comprise the composition according to the invention in an amount of 99.8% by weight, with 0.1% by weight of vitamin D3 in the form of cholecalciferol and 0.1% by weight of vitamin K in the form of menaquinone-7. The composition may preferably contain 97.1% by weight of MCT oil, 2.8% by weight of silicic acid and 0.1% by weight of Bacillus subtilis strain MM 40.

The cosmetic product can comprise the composition according to the invention in an amount of 35.4% by weight, and can also contain 63.8% by weight of almond oil, 0.5% by weight of vitamin E and 0.3% by weight of coriander essential oil. The composition may preferably contain 98.9% by weight of MCT oil, 1.0% by weight of silicic acid and 0.1% by weight of Bacillus subtilis strain MM 40.

In another advantageous embodiment, the cosmetic product can comprise the composition according to the invention in an amount of 70% by weight, additionally containing 15% by weight olive oil, 10% by weight mint essential oil and 5% by weight oregano essential oil. The composition may preferably contain 97.1% by weight of MCT oil, 2.8% by weight of silicic acid and 0.1% by weight of Bacillus subtilis strain MM 40.

Particularly preferred minerals are calcium and magnesium salts selected from the group consisting of calcium carbonate, calcium chloride, calcium salts of citric acid, calcium gluconate, calcium glycerophosphate, calcium lactate, calcium salts of orthophosphoric acid, calcium hydroxide, calcium oxide, magnesium acetate, magnesium carbonate, magnesium chloride, magnesium salts of citric acid, magnesium gluconate, magnesium glycerophosphate, magnesium salts of orthophosphoric acid, magnesium lactate, magnesium hydroxide, magnesium oxide and magnesium sulfate, iron oxide, potassium oxide, sodium selenite, selenomethionine and mixtures thereof.

In a further advantageous embodiment, the food supplement can comprise the composition according to the invention in an amount of 23% by weight, and can also contain 51.8% by weight calcium, 24.6% by weight magnesium and 0.6% by weight hydroxypropyl methylcellulose. The composition may preferably contain 92.9% by weight of MCT oil, 7.0% by weight of silicic acid and 0.1% by weight of Bacillus subtilis strain MM 40.

In a further advantageous embodiment, the dietary supplement can comprise the composition according to the invention in an amount of 23% by weight, with 51.7% by weight calcium, 24.5% by weight magnesium, 0.6% by weight hydroxypropyl methylcellulose, 0.1% by weight-% vitamin D3 in the form of cholecalciferol, and 0.1% by weight of vitamin K in the form of menaquinone-7. The composition may preferably contain 92.9% by weight of MCT oil, 7.0% by weight of silicic acid and 0.1% by weight of Bacillus subtilis strain MM 40.

Furthermore, the food supplement and/or the cosmetic product can contain food additives such as, in particular, colorants, preservatives and/or antioxidants. These are used to improve the texture, smell, color and chemical and microbiological stability of the product in question.

Preservatives can be selected, for example, from the group of sorbic acid, potassium sorbate, calcium sorbate, benzoic acid, sodium benzoate, potassium benzoate, calcium benzoate, ethyl 4-hydroxybenzoic acid, sodium ethyl 4-hydroxybenzoate, methyl 4-hydroxybenzoate, sulfur dioxide, sodium sulfite, sodium hydrogen sulfite, sodium disulfite, potassium disulfite, calcium disulfite, calcium bisulfite, potassium bisulfite, malic acid, fumaric acid and lysozyme and mixtures thereof.

Antioxidants can be selected, for example, from the group consisting of ascorbic acid, sodium ascorbate, calcium ascorbate, ascorbyl palmitate, ascorbyl stearate, alphatocopherol, gamma-tocopherol, delta-tocopherol, propyl gallate, octyl gallate, dodecyl gallate, isoascorbic acid, sodium isoascorbate, tert-butylhydroquinone, citric acid, phosphates, phosphoric acid, succinic acid and rosemary extract and mixtures thereof.

Furthermore, the food supplement and/or cosmetic product according to the invention can comprise aromas in order to give the product in question a special smell and/or taste. Aromas can be selected, for example, from the group consisting of natural aromas, aroma extracts, thermally obtained reaction aromas, smoke aromas, aroma precursors and other aromas or mixtures thereof. The food supplement and/or the cosmetic product preferably includes flavorings with a fruity flavor, for example selected from the series comprising oranges, blood oranges, grapefruit, tangerines, lemons and kumquats. Oranges and lemons are particularly preferred, especially as lemon oil or orange oil. Other favorite types include berries, blueberries, raspberries, strawberries, apple, pear, grapes, melon, coriander, sandalwood, fennel, frankincense, and ginger. Furthermore, vanilla and chocolate flavors can be preferred flavors.

The food supplement according to the invention and/or the cosmetic product is advantageously free of aluminum.

### EXAMPLES

The invention and the technical environment are explained in more detail below using examples. It should be noted that the invention should not be limited by the examples shown.

### Example 1

To test the antimicrobial effect of Bacillus subtilis strain MM 40 (DSM 21097), MH agar (Müller-Hinton-Agar) was used and inoculated with MRSA bacteria from the database of the University Hospital Lubeck (UKSH Lubeck). Bacillus subtilis DSM 33619 (DSM 21097) was applied using sterile test discs.

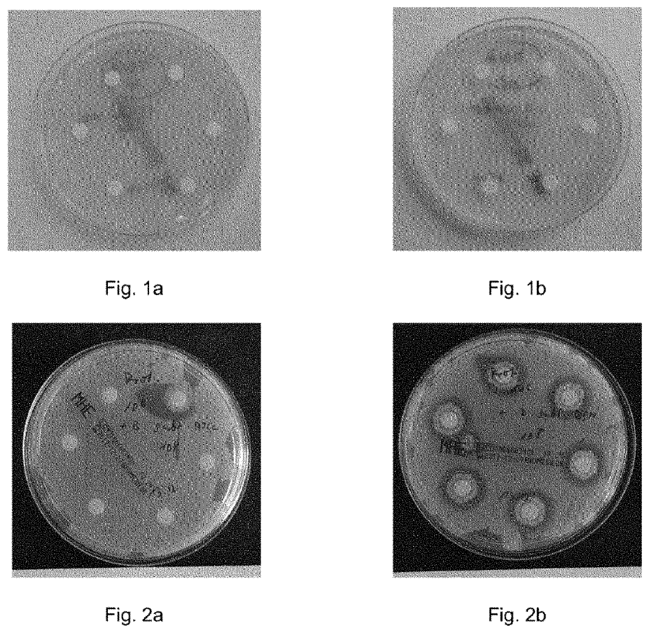

As can be seen from the illustration in FIG. 1b, an inhibition zone around the MRSA bacteria could be identified. The ATCC 6633 strain of Bacillus subtilis, which had no inhibition zone (FIG. 1a), was used as a control.

Furthermore, a comparison with the pathogenic bacteria “Proteus vulgaris” was carried out using the same method. An inhibition zone around the bacteria Proteus vulgaris was also found (FIG. 2b). The ATCC 6633 strain of Bacillus subtilis, which had no inhibition zone (FIG. 2a), was used as a control.

### Example 2

To investigate the proliferation rate of the bacterium Bacillus subtilis strain MM 40 (DSM 21097), a composition comprising 97.1% by weight MCT oil, 2.8% by weight silica and 0.1% by weight Bacillus subtilis strain MM 40 was ground together and the propagation rate thereof was examined in accordance with DIN EN ISO 4833-2:2014-05, § 64 LFGB L 00.00-88/2:2015-06. This showed an increase in the CFUs from 1*10⁵ CFU per mL of the composition to 4*10¹⁰ CFU per mL of the composition.

A comparison composition without the silica showed an increase rate of only 1*10⁵ CFU per mL of composition to 1*10⁷ CFU per mL of composition.

1. A composition comprising:
at least 70% by weight of a vegetable oil and/or fat substance,
at least 0.5% by weight silica, and
at least 0.05% by weight of Bacillus subtilis selected from any one of the strains comprising LA13030, CUL HU58, DE 111, DSM 15544, PXN 21, Rosell-179 and/or DSM 33619.

2. The composition according to claim 1, comprising at least 80% by weight of the vegetable oil and/or fat substance.

3. The composition according to claim 1, comprising at least 0.8% by weight, silicic acid.

4. The composition according to claim 1, wherein the silicic avid is a precipitated and/or a fumed silicic acid.

5. The composition according to claim 1, wherein the vegetable oil and/or fat substance is selected from the group consisting of olive oil, coconut oil, almond oil, pomegranate seed oil, mango butter, shea butter and/or mixtures thereof.

6. The composition according to claim 1, wherein the vegetable oil and/or fat substance is selected from the series of medium-chain triglycerides (MCT).

7. A method of preparing a composition according to claim 1, comprising the steps of:
preparing a composition comprising at least 70% by weight of a vegetable oil and/or fat substance, at least 0.5% by weight of silicic acid, and at least 0.05% by weight of Bacillus subtilis selected from any one of the strains comprising LA13030, CUL HU58, DE 111, DSM 15544, PXN 21, Rosell-179 and/or DSM 33619, and
mixing the components so that the composition is obtained.

8. The method according to claim 7, wherein the silica is dried prior to mixing.

9. A food supplement comprising a composition according to claim 1.

10. The food supplement according to claim 9, wherein this is free from aluminum.

11. A cosmetic product comprising a composition according to claim 1.

12. The composition according to claim 1, comprising at least 90% by weight of the vegetable oil and/or fat substance.

13. The composition according to claim 1, comprising at least 92% by weight of the vegetable oil and/or fat substance.

14. The composition according to claim 1, comprising at least 97% by weight of the vegetable oil and/or fat substance.

15. The composition according to claim 1, comprising at least 99% by weight of the vegetable oil and/or fat substance.

16. The composition according to claim 1, comprising preferably at least 1.0% by weight silicic acid.

17. The composition according to claim 1, comprising preferably at least 2.5% by weight silicic acid.

18. The composition according to claim 1, comprising preferably at least 2.8% by weight silicic acid.

19. The composition according to claim 1, comprising preferably at least 7.0% by weight silicic acid.
